(19) United States
(12) Patent Application Publication
(10) Pub. No.: US 2021/0113751 A1
(43) Pub. Date: Apr. 22, 2021

(54) PULSATILE BLOOD PUMP WITH ACTIVE ELEMENT AND THROMBUS RINSE

(71) Applicant: HeartWare, Inc., Miami Lakes, FL (US)

(72) Inventors: Fernando CASAS, Miami Lakes, FL (US); Mustafa Ertan TASKIN, Cooper City, FL (US); David A. SCHAFIR, Miami Shores, FL (US)

(21) Appl. No.: 16/655,628

(22) Filed: Oct. 17, 2019

(51) Int. Cl. A61M 1/10

(52) U.S. Cl. CPC A61M 1/1005; A61M 1/1058

(57) ABSTRACT

An implantable blood pump includes a housing defining an inlet and an outlet and a flow path therethrough. A rotor is disposed within the housing. A stator is disposed within the housing, the stator being configured to rotate the rotor when a current is applied to the stator. A volute is disposed distal to the rotor proximate the outlet, the volute including a tongue composed of a piezoelectric material.

### CROSS-REFERENCE TO RELATED APPLICATION

n/a

### FIELD

The present technology is generally related to implantable blood pumps having an active element.

### BACKGROUND

Implantable blood pumps used as mechanical circulatory support devices or “MCSDs” include a pumping mechanism to move blood from the heart out to the rest of the body. The pumping mechanism may be a centrifugal flow pump, such as the HVAD® Pump manufactured by HeartWare, Inc. in Miami Lakes, Fla., USA. The HVAD® Pump is further discussed in U.S. Pat. No. 8,512,013, the disclosure of which is hereby incorporated herein in its entirety. In operation, the blood pump draws blood from a source such as the right ventricle, left ventricle, right atrium, or left atrium of a patient's heart and impels the blood into an artery such as the patient's ascending aorta or peripheral artery.

In an exemplary HVAD® pump, an impeller is positioned within a housing having an upstream inflow cannula and a downstream outlet proximate a volute. The impeller is configured to rotate along an axis defined by the rotor and to impel blood upstream from the inflow cannula downstream to the outlet. In such a configuration, the impeller pumps blood in a direction substantially perpendicular to the axis about which it rotates. Dual stators are included in the pump, one upstream of the impeller and one downstream from the impeller and are each configured to rotate the impeller to impel blood. Disposed between the impeller and each respective stator is a non-ferromagnetic ceramic disk that separates the respective stator from the impeller and provides a smooth surface to pump blood.

In another configuration, the pumping mechanism may be an axial flow pump which supports various flow types, such as the MVAD® Pump, also manufactured by HeartWare, Inc. in Miami Lakes, Fla., USA. The MVAD® Pump is further discussed in U.S. Pat. Nos. 8,007,254 and 9,561,313 and U.S. Patent App. No. 15/475,432, filed Mar. 31, 2017, the disclosure of which is hereby incorporated in its entirety. Blood flowing within the MVAD® Pump, like other MCSDs, is also subject to thrombus and infection.

### SUMMARY

The techniques of this disclosure generally relate to implantable blood pumps having an active element.

In one aspect, the present disclosure provides an implantable blood pump includes a housing defining an inlet and an outlet and a flow path therethrough. A rotor is disposed within the housing. A stator is disposed within the housing, the stator being configured to rotate the rotor when a current is applied to the stator. A volute is disposed distal to the rotor proximate the outlet, the volute including a tongue composed of a piezoelectric material.

In another aspect, the volute and the tongue are composed of different materials.

In another aspect, the tongue is deformable when a voltage is applied to the tongue.

In another aspect, the tongue is deformable toward and away from a longitudinal axis defined by the flow path.

In another aspect, the rotor is configured to pump blood along the flow path toward the volute.

In another aspect, the housing includes an inflow cannula disposed about the flow path, the inflow cannula defining a proximal end and a distal end, and wherein the inflow cannula defines the inlet at its proximal end.

In another aspect, the tongue is electrically coupled to a voltage source.

In one aspect, a method of creating pulsatile flow in a patient having an implantable blood pump, the implantable blood pump having a volute, the volute having a tongue composed of a piezoelectric material, includes applying a voltage to the tongue for a predetermined period of time during operation of the blood pump.

In another aspect, the application of the voltage to the tongue occurs continually at predetermined internals.

In another aspect, the implantable blood pump is an axial flow pump.

In another aspect, the implantable blood pump includes a rotor configured to pump blood, and wherein the method further includes reducing a predetermined set speed of the rotor to a reduced speed during the application of the voltage to the tongue.

In one aspect, an implantable blood pump system includes an implantable blood pump, the implantable blood pump including a rotor configured to pump blood and a volute downstream of the rotor, the volute including a tongue composed of a piezoelectric material. A controller is in communication with the implantable blood pump, the controller being configured to power the implantable blood pump and to apply a voltage to the tongue.

In another aspect, the controller is implantable within a body of a patient.

In another aspect, the controller is further configured to continually apply the voltage to the tongue for a predetermined period of time during operation of the blood pump.

In another aspect, the controller is further configured to reduce a predetermined set speed of the rotor to a reduced speed during the application of the voltage to the volute.

In another aspect, the controller is configured to increase the reduced speed to the predetermine set speed when the voltage is not applied to the tongue.

In another aspect, the tongue is deformable.

In another aspect, the implantable blood pump defines a major longitudinal axis, and wherein the tongue is deformable toward and away from the major longitudinal axis.

In another aspect, the implantable blood pump is at least one from the group consisting of an axial flow pump and a centrifugal pump.

In another aspect, the controller is further configured to apply the voltage to the tongue continually at predetermined intervals.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is schematic view of an implantable blood pump system constructed in accordance with the principles of the present application;

FIG. 2 is an exploded view of the implantable blood pump shown in FIG. 1;

FIG. 3 is a top inside view of the volute shown in FIG. 2, with the tongue deformed at different degrees;

FIG. 4 is a top inside view of the volute shown in FIG. 2 with a normal tongue position and a deformed tongue position showing the associated flow and pressure through the volute for a pump operating at the same speed; and

FIG. 5 is a graph of HQ curves for a pump operating at different speeds and different tongue positions.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

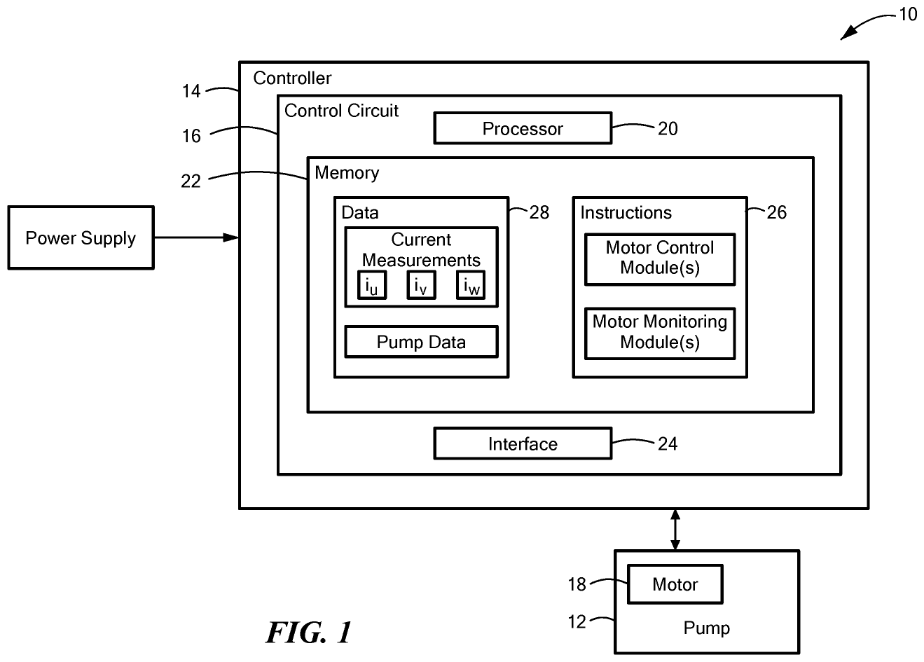

Referring now to the drawings in which like reference designators refer to like elements there is shown in FIG. 1 a block diagram of an exemplary system 10 constructed in accordance with the principles of the present application and designated generally “10.” The system 10 includes an implantable blood pump 12 in communication with a controller 14. The blood pump 12 may be the MVAD® Pump or another mechanical circulatory support device fully or partially implanted within the patient. The controller 14 includes a control circuit 16 having control circuitry for monitoring and controlling startup and subsequent operation of a motor 18 implanted within the blood pump 12. The controller 14 may also include a processor 20 having processing circuitry, a memory 22, and an interface 24. The memory 22 stores information accessible by the processor 20 and processing circuitry, including instructions 26 executable by the processor 20 and/or data 28 that may be retrieved, manipulated, and/or stored by the processor 20. The blood pump 12 may be a continuous flow blood pump, such as, without limitation, the MVAD® pump referenced above, and may include a housing having a rotor therein and a volute as discussed in more detail below.

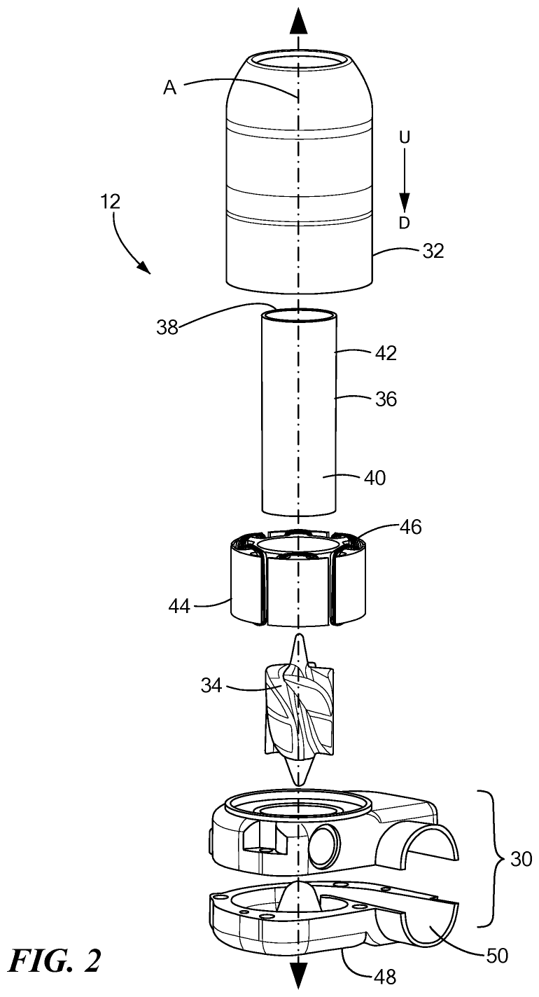

FIG. 2 is an exploded view of the blood pump 12 include a housing 30 having an inlet cannula 32 and a rotor 34 such as an impeller, proximate the inlet cannula 32 to impel the blood. The inlet cannula 32 includes an inner tube 36 formed from a non-magnetic material, such as a ceramic. The inner tube 36 includes an interior surface 38 defining a cylindrical bore 40 for receiving the rotor 34 therein. The inner tube 36 also includes a cylindrical outer surface 42 surrounded by a stator 44 having one or more coils 46. A voltage is applied to the coils 46 from a drive circuit (not shown) to produce an electromagnetic force to rotate the rotor 34. In particular, the electromagnetic force of the coils 46 exhibits an electromagnetic field which interacts with a magnetic field of the rotor 34 to suspend the rotor 34 within the cylindrical bore 40 and rotate the rotor 34. In addition to or in lieu of the magnetic forces, the rotor 34 may be suspended within the housing 30 using one or more hydrodynamic forces.

Rotation of the rotor 34 impels the blood along a fluid flow path from an upstream direction U to a downstream direction D through the inner tube 36 toward a volute 48 and then out through and outlet 50, through which the blood is expelled. The fluid flow path may be referred to as a blood flow path. Further details associated with rotary blood pumps are described in U.S. Pat. No. 8,007,254, the disclosure of which is incorporated herein by reference in the entirety. The blood pump 12 defines a housing axis “A” extending therethrough and along the fluid flow path from the upstream to the downstream direction. The rotor 34 moves in an axial direction relative to the housing 30 along the housing axis. When fluid, such as blood, passes through the blood pump 12, the fluid imparts a thrust on the rotor 34 which causes the rotor 34 to move. A magnitude of the thrust is related to the fluid flow rate through the blood pump 12. In other words, the axial position of the rotor 34 relative to the housing 30 is proportional to the fluid flow rate through the blood pump 12, which is proportional to the thrust.

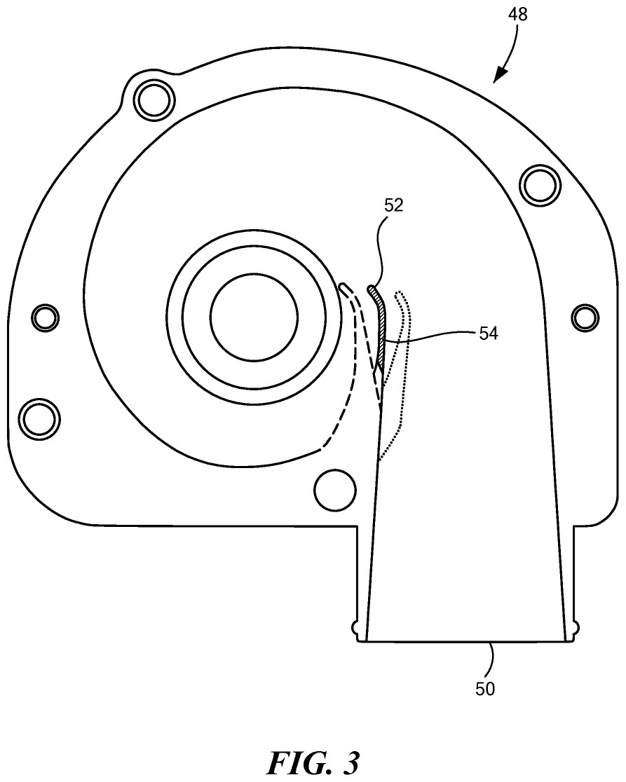

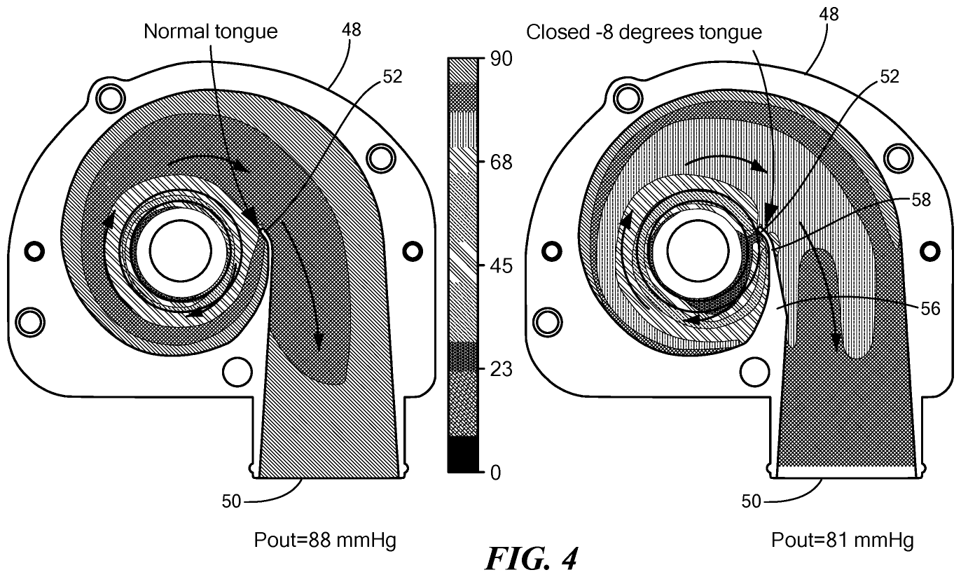

Referring now to FIGS. 3-4, the volute 48 includes a tongue 52 in direct contact with blood flowing through the volute 48. The tongue 52 projects from the volute 48 and, in part, defines the flow path of blood through the volute 48 toward the outlet 50. In an exemplary configuration, the tongue 58 includes, at least in part, a piezoelectric material 54 that is configured to deform the tongue 52 in response to an applied electric field. In one configuration, the entirety of the tongue 52 is composed of a piezoelectric material 54, or alternatively, may be coated with it a piezoelectric material 54, for example, a piezoelectrical crystal. In another configuration, the piezoelectric material 54 may be disposed on the tongue 52 and coated or otherwise encased in a corrosion resistant material, for example, Nitinol or other flexible materials that flex when the piezoelectrical material deforms the tongue 52. The piezoelectric material 54 may be hard wired to a power source exterior to the pump 12 for example, underneath the volute 48 or alternatively may have its own integrated power source. In other configurations, the piezoelectric material 54 may be included in a MEMS device adhered to a portion of the tongue 52. Such a MEMS device may have its own integrated power surface to apply the electric field, or alternatively, may be hard wired into the power source that powers the pump 12. In a configuration where the entirety of the tongue 52 includes the piezoelectric element 54, a portion or the entirety of the tongue 52 may be deformable in a direction toward or away from the flow path axis A. For example, as shown in FIG. 3, the tongue 52 may be deformed in a ranged from −20 degrees to +20 degrees toward and away from the flow path axis A as shown by the dashed lines.

Referring now to FIG. 4, the controller 14 may control the time when the electric potential is applied to the tongue 52, which may be periodic for a predetermined period of time or on demand to cause the deformation. When applied periodically, the deformation of the tongue 52 may cause a pulsatile effect on the blood flow out through the volute 48 without changing the overall flow or pressure profile of the pump 12. In other words, to overcome the pressure in the blood vessel receiving the blood flow from the pump 12, approximately 90 mmHG of pressure at the outlet is desirable. By temporarily and periodically lowering the outlet pressure to approximately 80 mmHg, this may create a washing effect and pulsatility without effecting the flow profile of the pump 12. The arrows and shading on the graph shown in FIG. 4 indicate flow direction and pressure respectively. Both pumps shown in FIG. 4 used the same fluid and pump speed.

In one configuration, only a portion of the tongue 52 includes the piezoelectric element 54. For example, the tongue 52 includes a proximal portion 56 coupled to the volute 48 and distal portion 58 extending away from the volute 48. The distal portion 56 may include the piezoelectric element 58 and may deform toward the fluid flow path axis A or away from it. For example, as shown in FIG. 4 the tongue 52 deforms toward the fluid flow axis A lowering the pressure of the blood flow at the outlet may create a washing effect which may prevent the formation of thrombus or otherwise dislodge thrombus from the pump 12.

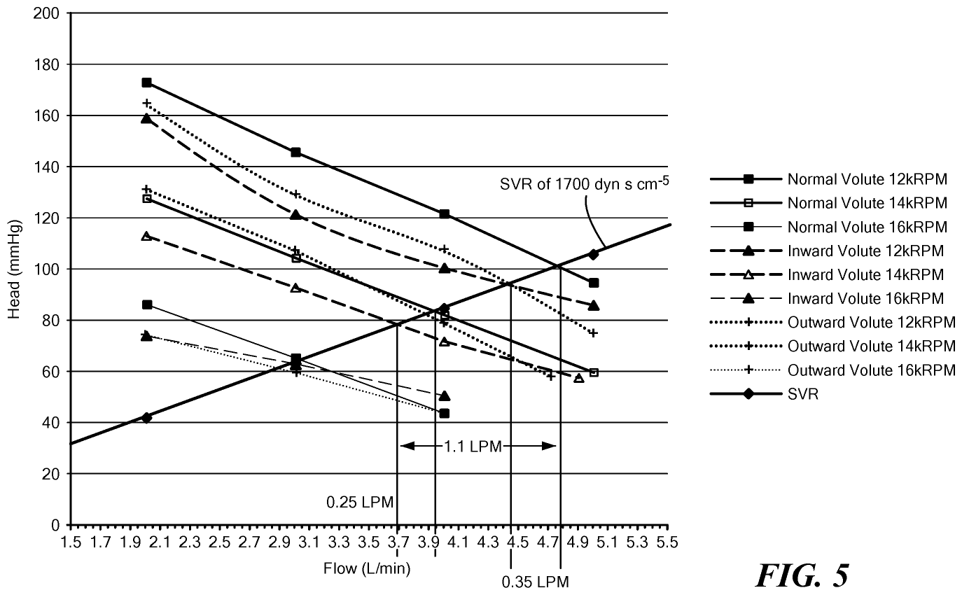

Referring now to FIG. 5, which shows the HQ performance of three tongue 52 locations, neutral, +8 degrees outward, and −8 degrees inward. A combination of deforming the tongue 52 with a reduction in pump speed, for example, by 2 k RPM, changes the flow by 1.1 LPM. Thus, deformation of the tongue 52 can introduce pulsatility into the system without speed modulation.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An implantable blood pump, comprising
a housing defining an inlet and an outlet and a flow path therethrough;
a rotor disposed within the housing;
a stator disposed within the housing, the stator being configured to rotate the rotor when a current is applied to the stator; and
a volute disposed distal to the rotor proximate the outlet, the volute including a tongue composed of a piezoelectric material.

2. The implantable blood pump of claim 1, wherein the volute and the tongue are composed of different materials.

3. The implantable blood pump of claim 1, wherein the tongue is deformable when a voltage is applied to the tongue.

4. The implantable blood pump of claim 3, wherein the tongue is deformable toward and away from a longitudinal axis defined by the flow path.

5. The implantable blood pump of claim 1, wherein the rotor is configured to pump blood along the flow path toward the volute.

6. The implantable blood pump of claim 1, wherein the housing includes an inflow cannula disposed about the flow path, the inflow cannula defining a proximal end and a distal end, and wherein the inlet is defined at the proximal end.

7. The implantable blood pump of claim 1, wherein the tongue is electrically coupled to a voltage source.

8. A method of creating pulsatile flow in a patient having an implantable blood pump, the implantable blood pump having a volute, the volute having a tongue composed of a piezoelectric material, comprising:
applying a voltage to the tongue for a predetermined period of time during operation of the blood pump.

9. The method of claim 8, wherein applying the voltage to the tongue occurs continually at predetermined internals.

10. The method of claim 8, wherein the implantable blood pump is at least one from the group consisting of an axial flow pump and a centrifugal pump.

11. The method of claim 8, wherein the implantable blood pump includes a rotor configured to pump blood, and wherein the method further includes reducing a predetermined set speed of the rotor to a reduced speed during the application of the voltage to the tongue.

12. An implantable blood pump system, comprising:
an implantable blood pump, the implantable blood pump including a rotor configured to pump blood and a volute downstream of the rotor, the volute including a tongue composed of a piezoelectric material; and
a controller in communication with the implantable blood pump, the controller being configured to power the implantable blood pump and to apply a voltage to the tongue.

13. The system of claim 12, wherein the controller is implantable within a body of a patient.

14. The system of claim 12, wherein the controller is further configured to continually apply the voltage to the tongue for a predetermined period of time during operation of the blood pump.

15. The system of claim 12, wherein the controller is further configured to reduce a predetermined set speed of the rotor to a reduced speed during the application of the voltage to the volute.

16. The system of claim 15, wherein the controller is configured to increase the reduced speed to the predetermine set speed when the voltage is not applied to the tongue.

17. The system of claim 12, wherein the tongue is deformable.

18. The system of claim 17, wherein the implantable blood pump defines a major longitudinal axis, and wherein the tongue is deformable e toward and away from the major longitudinal axis.

19. The system of claim 12, wherein the implantable blood pump is an axial flow pump.

20. The system of claim 12, wherein the controller is further configured to apply the voltage to the tongue continually at predetermined intervals.
